(19) United States
(12) Patent Application Publication
(10) Pub. No.: US 2013/0316919 A1
(43) Pub. Date: Nov. 28, 2013

(54) BIOMARKERS FOR THE DIAGNOSIS OF MULTIPLE SCLEROSIS

(71) Applicants: Paolo Edomi, Trieste (IT); Sara Bembich, Trieste (IT); Andrea Cortini, Cervignano Del Friuli (IT)

(72) Inventors: Paolo Edomi, Trieste (IT); Sara Bembich, Trieste (IT); Andrea Cortini, Cervignano Del Friuli (IT)

(21) Appl. No.: 13/992,625

(22) PCT Filed: Dec. 12, 2011
(86) PCT No.: PCT/EP2011/072440

§ 371 Date: Jun. 7, 2013

(30) Foreign Application Priority Data
Dec. 10, 2010 (IT) PD2010A000374

(51) Int. Cl. G01N 33/68

(52) U.S. Cl. CPC G01N 33/6893
USPC 506/2; 436/501; 506/9; 435/7.92

(57) ABSTRACT

The invention relates to biological markers for use in the diagnosis of multiple sclerosis and the use of said markers for distinguishing between patients with multiple sclerosis and patients with other neurological diseases. The invention further relates to a diagnostic technique for multiple sclerosis using said biological markers.

### FIELD OF THE INVENTION

The invention relates to biological markers for use in the diagnosis of multiple sclerosis and the use of said markers for distinguishing between patients with multiple sclerosis and patients with other neurological diseases. The invention further relates to a diagnostic technique for multiple sclerosis using said biological markers.

### PRIOR ART

Multiple sclerosis (MS) is regarded as the prototype of inflammatory autoimmune diseases of the central nervous system (CNS). MS is the commonest neurological pathology in young adults, affecting more than a million individuals world-wide, including 400,000 in Europe and nearly 60,000 in Italy, with highest incidence in Sardinia (1 in 700 inhabitants) and causes substantial invalidity of 50% of patients. The characteristic feature of the disease is demyelination plaque. It is known that the development of the plaque of the lesion involves an initial inflammatory phase, followed by a progressive chronic phase, and although the individual stages have not yet been fully elucidated, most of the evidence is in favour of an autoimmune pathogenesis. The factor that triggers the disease is still unknown, but the most widely held view is that the autoimmune reaction depends on a new contact with environmental factors, such as viruses, which mimic molecules of self (molecular mimicry) (Svejgaard et al., 2008).

While the role of the T cells in the pathogenesis of MS is well known, the role of the B cells and autoantibodies is largely unsolved, although their importance is becoming more and more evident (Klawiter and Cross, 2007). At the same time, the importance of identifying biological markers of MS is growing steadily, especially because of the heterogeneity of patients' immune response (Reindl et al., 2006).

There are various indications that there is a link between antibodies, both of class G and of class M, and the disease. B cells and specific myelin autoantibodies are present in the plaques of patients with MS, and an increase in the production of immunoglobulins (Ig) in the cerebrospinal fluid has been observed in more than 90% of patients with MS. More recently, the presence of structures similar to lymphatic follicles has been observed in the meninges of some patients with MS and this has been linked to more acute demyelination (Magliozzi et al., 2007). Further complications result from the phenomenon of “epitope spreading”, by which the autoimmune response spreads from the single initial component of an affected tissue to other autoantigens. The progression of murine experimental autoimmune encephalomyelitis (EAE) and of MS is accompanied by a decline in autoreactivity of the primary T cells and concomitant appearance of new autoreactivity simultaneous with the tissue damage mediated by the autoimmune response (McMahon et al., 2005).

The specificity of the epitopes recognized by the anti-myelin antibodies in MS is still an open question. Various epitopes of myelin proteins have occasionally been identified as immunodominant in patients with MS with relapses and remissions (Khalil et al., 2006).

This variability might indicate that identification of the autoantigens involved in triggering or perpetuating MS may largely depend on the system with which they are sought. Whereas all the epitopes of the T cells are linear, an autoantibody specific to myelin proteins can bind a conformational or linear epitope (Dharmasaroja, 2003). Antibodies, especially IgG, that bind conformational epitopes of the extracellular domain of MOG with high affinity were found in patients' serum. However, these pathogenic antibodies have not yet been characterized in humans (Lalive et al., 2006).

Determination of the specificity of the epitopes is of considerable diagnostic value. Whereas the data from conventional magnetic resonance enable clinicians to identify the disease and the particular phase, there are no accepted biological indicators for diagnosing the activity of the disease in MS (Saizer et al., 2010).

Therefore there is still a need for identifying a biological marker that is of diagnostic value for multiple sclerosis.

Berger et al. (2003) showed that patients with the clinically isolated form of MS (CIS), seropositive for anti-MOG and anti-MBP antibodies, have a higher probability of having relapses compared with seronegative patients. Moreover, it was found that the titre of IgG, but not of IgM, specific to the native form of MOG, was significantly higher in patients with MS compared with a control group, with a higher prevalence for the patients with the primary progressive form (Zhou et al., 2006).

Conversely, Lim et al. (2005) showed for example that the levels of anti-myelin IgG are not correlated with the clinical parameters of the disease.

Using a cellular assay that measures in detail the antibodies directed against the human MOG protein expressed on the cell membrane, native IgGs specific to MOG were found more frequently in the serum of CIS and RR patients, only marginally in secondary progressive MS and not at all in primary progressive MS (Lalive et al., 2006). However, another study did not find any link between the presence of IgG and IgM anti-MOG and anti-MBP antibodies, detected by Western blot, and progression to clinically defined MS or a diagnosis of MS according to McDonald's criteria (Kuhle et al., 2007). Therefore the diagnostic value of serum antibodies to MOG and MBP for predicting a risk of progression to clinically defined MS in patients who have presented a clinically isolated syndrome is debatable at present (Polman and Killestein, 2007).

In the diagnosis of multiple sclerosis, it will be considered, in particular, that the main interest focuses on the possibility of discriminating between patients with neurological symptoms of various kinds, among various diseases affecting the nervous system, and patients with multiple sclerosis. Multiple sclerosis in fact has a multiplicity of symptoms that can be confused with those of other neurological diseases and sometimes diagnosis by means of nuclear magnetic resonance, the only system currently used, is not definitive (Ratchford and Calabresi, 2008). Consequently there is a need to identify markers that make it possible in particular to distinguish between patients with neurological symptoms, rather than between patients with multiple sclerosis and healthy subjects.

The aim of the present invention is therefore to identify a biological marker that would be of diagnostic value for multiple sclerosis and the use of said markers for distinguishing between patients with multiple sclerosis and patients with other neurological diseases.

A further aim is to identify an innovative system for searching for autoantigens in autoimmune diseases, and application thereof in the case of multiple sclerosis.

### SUMMARY OF THE INVENTION

The aforementioned aim was achieved with a protein or polypeptide selected from the group consisting of: SEQ ID NO. 2, SEQ ID NO. 3 and SEQ ID NO. 4 for use as biological marker for the diagnosis of multiple sclerosis.

Another aspect of the invention relates to a method for the diagnosis of multiple sclerosis using any protein selected from the group consisting of SEQ ID NO. 2, SEQ ID NO. 3 and SEQ ID NO. 4.

A further aspect of the present invention relates to a diagnostic kit for the diagnosis of an autoimmune disease comprising any protein selected from the group consisting of SEQ ID NO. 2, SEQ ID NO. 3 and SEQ ID NO. 4 that acts as antigen and a reagent for separate, simultaneous and successive use as a conjugated secondary antibody able to detect the antibodies that are present in biological samples and are reactive towards the protein or antigen used.

A further aspect of the invention relates to a method for searching for autoantigens in multiple sclerosis, comprising the steps of:

a. preparing a phage display library of scFv antibodies from the cerebrospinal fluid of one or more patients with multiple sclerosis;
b. preparing a phage display library enriched in ORF cDNA fragments from human brain;
c. selecting the library of step b with the library of step a, said selection being carried out by interacting the immobilized antibodies phage library onto an immunotube with the antigen phage library to the same phage titre;
d. sequencing the clones selected in step c.

### BRIEF DESCRIPTION OF THE DRAWINGS

The characteristics and advantages of the present invention will now be described in detail, referring to the appended FIGS. 1-9, and to the non-limiting examples supplied for purposes of illustration.

FIG. 1a: Schematic representation of the map of the vector pEP1 and the relevant sequence of the polylinker.

FIG. 1b: Schematic representation of the map of vector pEP2 and the relevant sequence of the polylinker.

FIG. 2: Diagram representing the procedural steps in construction of the antigen phage library indicating the oligonucleotides used.

FIG. 3: Schematic representation of the relative positions, with respect to the antigen identified by selection of the phage library: the human TCERG1 protein SEQ ID NO. 1 (Ag), of the recombinant protein: the polypeptide that corresponds to the portion between amino acids 677 and 1098 of the human TCERG1 protein SEQ ID NO. 2 (prot) and of the three peptides synthesized for validation of the respective diagnostic potentialities: polypeptide TCERG1A SEQ ID NO. 5 (pepA), polypeptide TCERG1 B SEQ ID NO. 6 (pepB) and polypeptide TCERG1C SEQ ID NO. 4 (pepC); the numbers indicated on the line underneath refer to the amino acids of the whole TCERG protein; the vertical bars indicate position of possible epitopes identified by homology with another antigen.

FIG. 4: Electrophoretic analysis of production of the recombinant protein TCERG1 (a.a. 677-1098) by acrylamide gel at 12.5% and Coomassie staining.

FIG. 4 (A): bacterial lysates of cells uninduced (lane 2) and induced for 3 (lane 3) and 16 hours (lane 4).

FIG. 4 (B): samples eluted after passage through column of streptactin: successive elution fractions (lanes from 2 to 5)

In lane 1: Molecular weight standards (Mark12, Invitrogen).

FIG. 4 (C): samples eluted after passage through column of histidines (C): successive elution fractions (lanes from 2 to 5).

In lane 1: Molecular weight standards (Mark12, Invitrogen).

FIG. 5: (A) “Scatter plot” representation of the reactivity of CSF samples from 17 patients with multiple sclerosis (MS) and 17 control subjects (OND) in comparison with the TCERG1 antigen carried by phage assayed by ELISA. The ratios of the values of optical density, relative to those obtained in the absence of antigen for each sample, are shown on the ordinate.

FIG. 5: (B) “Receiver Operating Characteristic” (ROC) graph for determining the diagnostic value of the antigen used in (A).

FIG. 6: (A) “Scatter plot” representation of the reactivity of serum samples from 20 patients with multiple sclerosis (MS) and 20 control subjects (OND) in comparison with the antigen TCERG1 as recombinant protein assayed by ELISA. The ratios of the optical density values relative to those obtained in the absence of antigen for each sample are shown on the ordinate.

FIG. 6: (B) ROC graph for determining the diagnostic value of the antigen used in (A).

FIG. 7: (A) “Scatter plot” representation of the reactivity of serum samples from 41 patients with multiple sclerosis (MS) and 40 control subjects (OND) in comparison with the peptide TCERG1-C assayed by ELISA. The ratios of the optical density values relative to those obtained in the absence of antigen for each sample are shown on the ordinate.

FIG. 7: (B) ROC graph for determining the diagnostic value of the antigen used in (A).

FIG. 8: (A) “Scatter plot” representation of the reactivity of serum samples from 30 patients with multiple sclerosis (MS), 40 control subjects (OND) and 10 healthy subjects (HD) in comparison with the peptide TCERG1-C assayed by ELISA. The ratios of the optical density values relative to those obtained in the absence of antigen for each sample are shown on the ordinate.

FIG. 8: (B) ROC graph for determining the diagnostic value of the antigen used in (A) relative to the MS and OND samples.

FIG. 9: Representation of competitive ELISA for recognition of the peptide TCERG1-C by a serum sample of a patient with multiple sclerosis and of a control subject. The samples were pre-incubated with increasing concentrations of peptide (μM, on the abscissa) and then assayed by ELISA; the values of optical density are given for each sample, the value of the negative control (absence of antigen) being equal for both sera (symbols: square=MS sample; diamond=control sample).

### DETAILED DESCRIPTION OF THE INVENTION

The invention therefore relates to a protein or polypeptide selected from the group consisting of: SEQ ID NO. 2, SEQ ID NO. 3 and SEQ ID NO. 4 for use as biological marker (or biomarker) for the diagnosis of multiple sclerosis.

For the purposes of the present invention, each protein or polypeptide corresponds to a sequence identified with a sequence number or SEQ ID NO. as given below:

SEQ ID NO. 1 corresponds to the amino acid sequence of the human TCERG1 protein TCERG1 transcription elongation regulator 1; Gene ID: 10915, NM_{—}006706.3→NP_{—}006697.2 transcription elongation regulator 1 isoform 1;

SEQ ID NO. 2: the amino acid sequence of the polypeptide that corresponds to the portion between amino acids 677 and 1098 of the human TCERG1 protein;

SEQ ID NO. 3: the amino acid sequence of the polypeptide that corresponds to the portion between amino acids 683 and 793 of the human TCERG1 protein;

SEQ ID NO. 4: the amino acid sequence of the polypeptide that corresponds to the portion between amino acids 751 and 770 of the human TCERG1 protein;

SEQ ID NO. 5: the amino acid sequence of the polypeptide that corresponds to the portion between amino acids 707 and 729 of the human TCERG1 protein;

SEQ ID NO. 6: the amino acid sequence of the polypeptide that corresponds to the portion between amino acids 730 and 750 of the human TCERG1 protein;

The proteins (polypeptides or peptides) can be used as antigens. Production of specific antibodies for the peptides identified makes it possible to carry out competitive assays in which the presence of antibodies specific to the antigen (auto-antibodies) in biological samples is detected by the capacity to sequester said antigen.

The proteins (polypeptides or peptides) can be produced in bacteria, in recombinant form obtained by techniques based on genetic engineering, or by chemical synthesis.

In cases in which chemical modifications or modifications by recombinant techniques (for example modifications such as acetylations, carboxylations, glycosylations, phosphorylations, amidations) do not alter the functionality of the proteins, are to be regarded as included in the present patent.

The marker identified and the respective consensus sequence, surprisingly, make it possible to distinguish between patients with MS and with other neurological diseases with a specificity of 90-98%. The sensitivity of 22-50% signifies that the antigen is potentially able to diagnose different states of the disease only to be found in certain patients.

Advantageously, another aspect of the present invention relates to a protein selected from the group consisting of: SEQ ID NO. 2, SEQ ID NO. 3 and SEQ ID NO. 4, as biological marker for monitoring the efficacy of therapeutic treatment in multiple sclerosis; and make it possible to monitor the parameters of the inflammatory state following treatment with interferon, and are correlated with the clinical state.

Until now, the main parameter of the efficacy of interferon treatment was reduction in the number of relapses. The advantage of the present invention is that it supplies biological markers for monitoring the efficacy of the treatment without having to wait to verify the relapses of the pathology.

The invention relates to a protein selected from the group consisting of: SEQ ID NO. 2, SEQ ID NO. 3 and SEQ ID NO. 4, as biological marker for distinguishing between patients with multiple sclerosis and patients with other neurological diseases such as polyneuropathies, polyneuritides, polyradiculoneuritides, encephalitides, myelitides, meningitides, leukoencephalopathies, vasculitides, Miller-Fisher and Guillain-Barré syndromes, amyotrophic lateral sclerosis, spastic tetraparesis, paraneoplastic neuropathies, Charcot-Marie-Tooth syndrome, spinal cord injuries, hydrocephalus, sub-arachnoid haemorrhages.

Surprisingly, in a preferred embodiment, the inventors of the present invention is made it possible to improve the possibility of discriminating between patients with neurological symptoms of various kinds, common to various diseases affecting the nervous system, and patients with multiple sclerosis. Multiple sclerosis in fact displays a multiplicity of symptoms that can be confused with those of other neurological diseases, and the diagnostic techniques currently used do not allow this distinction to be made. Consequently there is a need to identify markers that make it possible in particular to distinguish between patients with neurological symptoms, rather than between multiple sclerosis patients and healthy individuals.

According to another aspect of the invention, the protein selected from the group consisting of: SEQ ID NO. 2, SEQ ID NO. 3 and SEQ ID NO. 4, is a biological marker in protein platforms (arrays).

In a preferred embodiment, said protein platforms are miniaturized (protein chips). Miniaturization offers numerous advantages compared with the conventional techniques. These advantages comprise improvement in the accuracy and reproducibility of the data, shorter analysis times, minimum consumption of sample, potential for automation and integration of complex operational flows.

Another aspect of the invention relates to the protein selected from the group consisting of: SEQ ID NO. 2, SEQ ID NO. 3 and SEQ ID NO. 4, in which said protein is a biological marker for determining the individual immunological profiles.

These proteins can in fact, surprisingly, be used together with other polypeptides for constructing protein platforms (for example protein arrays) that are able to detect the presence of specific antibodies (auto-antibodies) and determine a patient's immune state and clinical profile.

In multiple sclerosis, it has been found to be very important to be able to discriminate between the various states of the disease. Another aspect of the invention relates to a protein selected from the group consisting of: SEQ ID NO. 2, SEQ ID NO. 3 and SEQ ID NO. 4, as biological marker for discriminating between different states of multiple sclerosis that are known to have different forms:
a. Relapsing-remitting form (RR);
b. Primary-progressive form (PP);
c. Secondary-progressive form (SP).

Another aspect of the invention relates to a method for the diagnosis of multiple sclerosis using any protein selected from the group consisting of SEQ ID NO. 2, SEQ ID NO. 3 and SEQ ID NO. 4.

A preferred method for the diagnosis of multiple sclerosis using any protein selected from the group consisting of SEQ ID NO. 2, SEQ ID NO. 3 and SEQ ID NO. 4 makes it possible to discriminate between patients with multiple sclerosis and patients with other inflammatory and non-inflammatory pathologies of the central nervous system or between patients before and after therapeutic treatment with interferon, which comprises the steps of:
a. providing a biological sample;
b. providing any protein selected from the group consisting of SEQ ID NO. 2, SEQ ID NO. 3 and SEQ ID NO. 4 immobilized on a solid substrate;
c. bringing the sample of step a. into contact with the substrate of step b.;
d. detecting the reaction.

The peptides or the protein corresponding to the marker identified as TCERG1 can be used in all assays of the diagnostic type in which samples of biological origin (serum, liquor) can be analysed for their reactivity to said marker.

In particular, this relates to all assays in which the biological marker selected from the group consisting of SEQ ID NO. 2, SEQ ID NO. 3 and SEQ ID NO. 4, can be used for identifying the presence of specific antibodies, i.e. capable of forming immune complexes with the marker, in biological samples. As an example, these antigen-antibody reactions can be detected with enzyme-linked immunosorbent assay (ELISA), radioimmunoassay (RIA), Western blot and LINE blot immunological assays, assays with protein microarrays.

Another aspect of the invention therefore relates to a method in which the diagnosis is performed by a technique selected from the group consisting of:
a. assays of the ELISA type;
b. assays of the radioimmunoassay (RIA) type;
c. assays of the Western blot and LINE blot type;
d. assays with protein microarrays.

Moreover, the protein sequences according to the present patent can be used for identifying the particular antigens present in biological samples by the production of antibodies specific to specified amino acid sequences. The immunological assays that can be used are similar to those enumerated above. Production of antibodies specific to the peptides identified makes it possible to carry out competitive assays in which the presence of antibodies specific to the antigen (auto-antibodies) in biological samples is detected by the capacity to sequester said antigen.

In a preferred method, control during execution of the assay takes place:
a. in the absence of antigen to detect the background of the reaction; and
b. in triplicate with a reference sample to be used as positive control, the value of which serves for standardizing the values of the biological samples under investigation.

The biological samples used in the present invention are preferably samples of serum or of cerebrospinal fluid.

A further aspect of the present invention relates to a diagnostic kit for the diagnosis of an autoimmune disease comprising any protein selected from the group consisting of SEQ ID NO. 2, SEQ ID NO. 3 and SEQ ID NO. 4 that acts as antigen and a reagent for separate, simultaneous and successive use as a conjugated secondary antibody able to detect the antibodies present in the biological samples and which are reactive to the protein or antigen used.

An autoimmune disease preferably diagnosed by means of the diagnostic kit of the present invention is Multiple Sclerosis.

The diagnostic kit makes it possible, surprisingly, to discriminate the patient with multiple sclerosis or a specified autoimmune state. The kit should envisage, for example, supply of the antigen immobilized on a solid substrate; detection of reaction with specific antibodies present in biological samples (e.g. ELISA) should take place in parallel with two types of controls: execution of the assay (1) in the absence of antigen to detect the background of the reaction and (2) in triplicate with a reference sample to be used as positive control, the value of which serves for standardizing the values of the samples under investigation.

A further aspect of the invention relates to a method for searching for autoantigens in multiple sclerosis, comprising the steps of:

a. preparing a phage display library of scFv antibodies from the cerebrospinal fluid of one or more patients with multiple sclerosis;
b. preparing a phage display library enriched in ORF cDNA fragments from human brain;
c. selecting the library of step b with the library of step a, said selection being carried out by interacting the immobilized antibodies phage library onto an immunotube with the antigen phage library to the same phage titre;
d. sequencing the clones selected in step c.

Another aspect of the invention relates to a process for searching for autoantigens in autoimmune diseases, comprising the steps of:

a. preparing a phage display library of scFv antibodies from biological samples of one or more patients with an autoimmune disease;
b. preparing a phage display library enriched in ORF cDNA fragments from human brain;
c. selecting the library of step b with the library of step a, said selection being carried out by interacting the immobilized antibodies phage library onto an immunotube with the antigen phage library to the same phage titre.
d. sequencing the clones selected in step c.

Until now, scFv from patients with MS have been used for selecting libraries of random peptides or of cDNA with the aim of identifying the antigens that guide the immune response in MS. scFvs cloned from patients' individual B cells have been used in very few cases, as they are very difficult to isolate.

This new selection procedure, obtained by interacting two phage libraries together, an antibody library and an antigen library, with a view to increasing the possibility of identifying rare autoantigens through the simultaneous manipulation of 10¹⁰-10¹² phage particles for each library. The antigen library that is produced is enriched with cDNA fragments coding for proteins expressed in the human brain.

Some examples of carrying out the invention and evaluating the benefits derived from the biomarker proteins of the invention are given below, as non-limiting examples illustrating said invention.

### EXAMPLES

### Example 1

### Biological Samples

The serum samples and cerebrospinal fluid were obtained after obtaining informed consent, and were stored at −80° C. All the patients with multiple sclerosis had been diagnosed according to the McDonald criteria, and for the most part had the RR form (with relapses and remissions); the clinical and personal data are summarized in Table 1. The patients with other neurological diseases comprised: polyneuropathies, polyneuritides, polyradiculoneuritides, encephalitides, myelitides, meningitides, leukoencephalopathies, vasculitides, Miller-Fisher and Guillain-Barré syndromes, amyotrophic lateral sclerosis, spastic tetraparesis, paraneoplastic neuropathies, Charcot-Marie-Tooth syndrome, spinal cord injuries, hydrocephalus, sub-arachnoid haemorrhages.

The samples were kindly supplied by the Multiple Sclerosis Centre of Trieste and Cagliari and by the Neurology Clinic of Padua.

TABLE 1
Clinical and personal data with respective mean values of
the patients with multiple sclerosis recruited for selection
of antigens and the following diagnostic validations.

| | | | Average age | | Average duration | |
|---|---|---|---|---|---|---|
| | | Female/ | (range) in | | (range) in | |
| MS samples | N | male | years | Diagnosis | years | EDSS range |
| Group 1 | 28 | 18/12 | 42.7 (21-66) | 20 RR | RR: 9.3 (3-28) | 0-3.5 (CIS |
| | | | | 7 CIS | CIS: 5 (4-7) | and RR) |
| | | | | 1 PP | PP: 27 | 8 (PP |
| Group 2 | 90 | 71/19 | 40.4 (19-63) | RR | 12 (1-36) | 0-6.5 |
| Group 3 | 18 | 7/11 | 48.7 (35-67) | SP | 18.9 (6-35) | 3-7 |
| Group 4 | 30 | 19/11 | 37.6 (22-54) | RR | 5.8 (1-20) | / |

### Bacterial Strains

The bacterial strains used were as follows: Escherichia coli DH5αF′ (Gibco BRL): F′/endA1 hsd17 (r_{K}⁻m_{K}⁺) supE44 thi-1 recA1 gyrA (Nal^{r}) re/A1Δ (lacZYA-argF) U169 deoR (F80dlacD-(lacZ)M15), for propagation of the phages and construction of the scFv (single-chain variable fragment) library; TOP10F′: F′{/acIqTn10(TetR)} mcrA Δ(mrr-hsdRMS-mcrBC)φ80lacZΔM15 ΔlacX74 recA1 araD139 Δ(ara-leu)7697 galU galK rpsL endA1 nupG, for constructing the cDNA library; BS1365: BS591 F″ Kan [BS591: recA1 endA1 gyrA96 thi-1 D lacU169 supE44 hsdR17 (lambdal mm434 nin5X1-cre)], for recombination of the library.

### Example 2

### Production of a Phage Display Library of scFv Antibodies from Cerebrospinal Fluid of Patients with MS

The next example describes obtaining a library of scFv antibodies by cloning of cDNA coding for variable regions of heavy (VH) and light (VL) antibody chains of two patients with multiple sclerosis.

The B cells were collected by centrifugation from the CSF of two RRMS patients; the total RNA was extracted and retrotranscribed into cDNA. The VH and VL regions were produced by PCR, assorted randomly and used for cloning in the pDAN5 vector as described by Sblattero et al. (2000). After transformation by electroporation into E. coli DH5αF′, a library of 2×10⁴ independent clones was obtained, with a diversity of 30.8% and 72.7% respectively for the VH and VL chains.

### Methodology:

Construction of the scFv Library from CSF

The RNA for constructing the scFv library was obtained from B cells of cerebrospinal fluid of two RR patients. Both patients were positive for the presence of oligoclonal bands and were not undergoing treatment; one was a woman of 40 years (start of the disease at 36) with an equal EDSS; the other was a man of 29 years (start of the disease at 27) with an EDSS equal to 3.5.

The total RNA was extracted from a pellet of 2×10⁴ B cells using the system PicoPure™ RNA Isolation Kit (Arcturus). The first cDNA strand was synthesized using random hexamers and SuperScript TM III RT (Invitrogen), according to the supplier's instructions. Each family of VH and VL genes was amplified separately in a final reaction volume of 20 μL containing 1U of ExTaq polymerase (TaKaRa) and the specific primer (Sblattero et al. 2000). In the case of the VH chains, the primer at 3′ was specific to the IgGs. PCR was conducted for 30 cycles with the following conditions: denaturation for 30 s at 94° C., pairing for 30 s at 55° C., extension for 45 s at 72° C. The individual VH and VL genes were combined to obtain two equimolar VH and VL mixtures, which were amplified and assembled as described by Sblattero et al. (2000).

The assembled PCR products coding for scFV were then mixed with a phagemid vector pDAN5 digested with BssHII/NheI enzymes at a molar ratio of 10:1 (fragments:vector) and submitted to ligation. The ligation mixture was transformed into DH5α F′ E. coli cells by electroporation. Individual clones obtained by the transformation were analysed by PCR, enzymatic fingerprinting and sequencing.

### Example 3

### Construction of the Vector by Selection of ORF Fragments

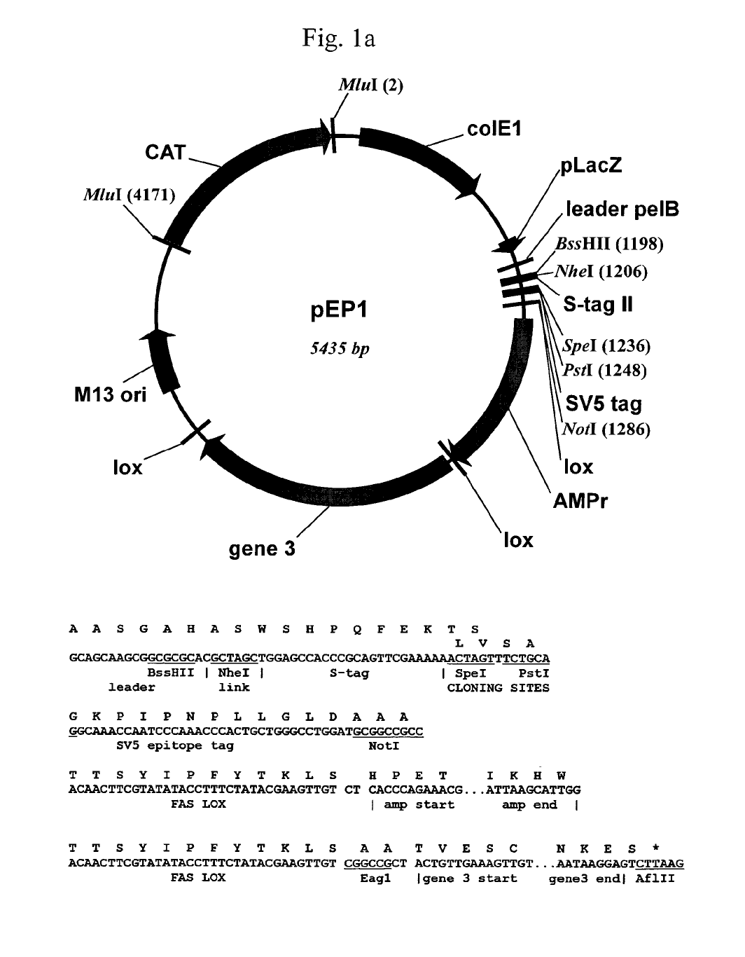

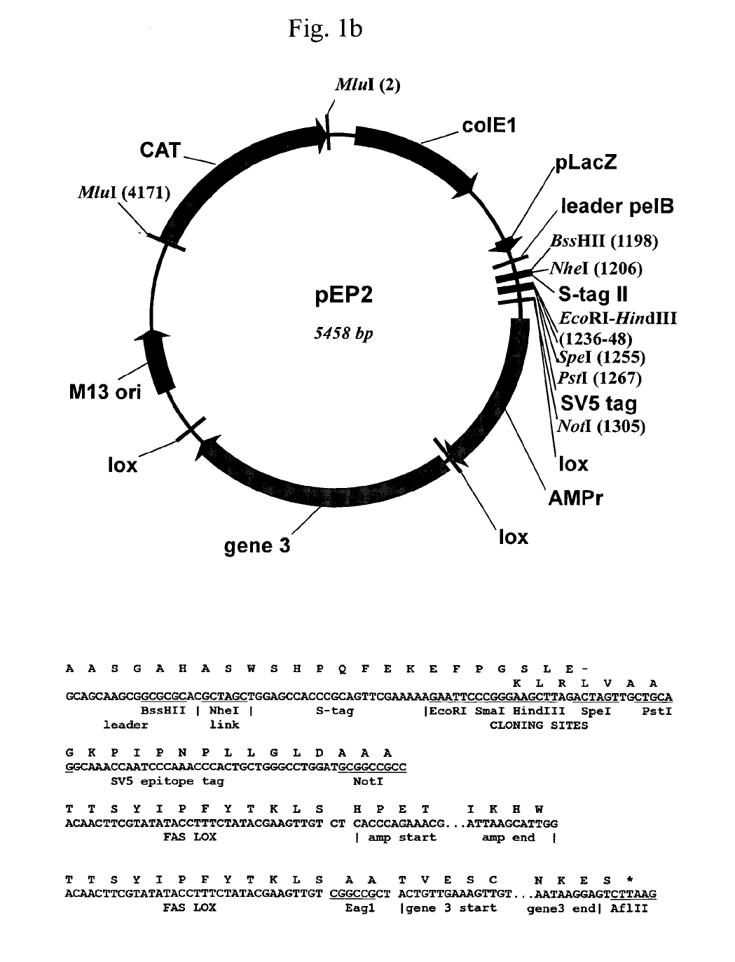

Starting from the skeleton of the vector pPAO2 (Zacchi et al., 2003), two new vectors were obtained, designated pEP1 and pEP2, by modifying the sequence of the polylinker. The essential characteristics of these vectors are shown in FIG. 1. The system for selecting ORF fragments by means of fusion with the β-lactamase gene was maintained.

The modifications introduced in the polylinker relate to: (a) the cloning strategy, as SpeI sites, were inserted for pEP1, and EcoRI and HindIII, for pEP2, by restriction-mediated cloning and (b) the system for purification and detection of the coded polypeptides (the sequence Streptag II [IBA-Go] was positioned upstream of the cloning site, replacing the existing tag sequences). In particular, the pEP2 vector allows oriented cloning of cDNA, according to the system OrientExpress Random Primers Novagen, to increase the probability of cloning fragments with the correct reading frame from 1/18 to 1/9. Moreover, the vectors have two new restriction sites for direct subcloning of ORF inserts into expression vectors and the amber stop codon (TAG), present in pPAO2 between cDNA insert and β-lactamase, was removed to make selection of ORF fragments more efficient.

### Methodology

Expression and Purification of scFv Antibodies

To produce recombinant scFv antibodies in soluble form, HB2151 cells were infected with phages of the antibody library from CSF and were grown at 37° C. in 2xYT medium containing 100 μg/mL of ampicillin up to an OD equal to 0.5. After adding IPTG to a final concentration of 0.5 mM, growth was continued overnight. After centrifugation at 4500 g for 20 min, the pellet was resuspended in 10 ml of lysis buffer (Tris-HCl 20 mM pH 8.0, NaCl 500 mM, imidazole 5 mM, Triton X100 0.1%) per gram of cells, together with lysozyme 100 μg/mL and DNase 30 μg/ml, and incubated in ice for 60 min. Then the samples were centrifuged at 4500 g for 20 min to separate the included bodies from the soluble cellular proteins. The included bodies containing the scFvs were resuspended in 10 ml of solubilizing buffer (Tris-HCl 20 mM pH 8.0, NaCl 500 mM, imidazole 5 mM, TritonX100 0.1% and urea 8M) and incubated for 1 hour at 4° C. The sample was centrifuged at 4500 g for 20 min and the scFvs were purified by affinity chromatography using NiNTA resin (IBA). Folding of the scFvs was carried out directly on the column using a linear gradient of urea from 8.0 M to 0 M. The renatured scFvs were eluted using a buffer containing Tris-HCl 20 mM pH 8.0, NaCl 500 mM and imidazole 300 mM.

### pEP Vectors

The phagemid pEP1 is derived from pPAO2 (Zacchi et al. 2003) modified in order to contain a new polylinker sequence. The new polylinker was cloned into pPAO2, digested with Bsshll and NotI, by insertion of two superposed oligonucleotides,

OLPE153
(5′-CGCGCACGCTAGCTGGAGCCACCCGCAGTTCGAAAAAACTAGTTTC
TGCAGGCA-3′)
and
OLPE135
(5′-GGCCGCATCCAGGCCCAGCAGTGGGTTTGGGATTGGTTTGCCTGCA
GAAACTAG-3′).

In particular, the sequence for StreptagII was introduced, and the restriction sites for EcoRI and SpeI.

The phagemid pEP2 was obtained from pEP1 in order to permit directed cloning.

A new polylinker sequence was inserted using the oligonucleotides

Poly1
(5′-AGCTCGGGTCTCGAGCTAGCCAAATTCTATTTCAAGGAG-3′)
and
Poly2
(5′-CCGGGCTGCAGCAACTAGTCTAAGCTTCCCGGGAATTCTTTTTCGA
ACTGCGGGTGGCTC-3′).

### Example 4

### Construction of a Phage Display Library Enriched in ORF cDNA Fragments from Human Brain

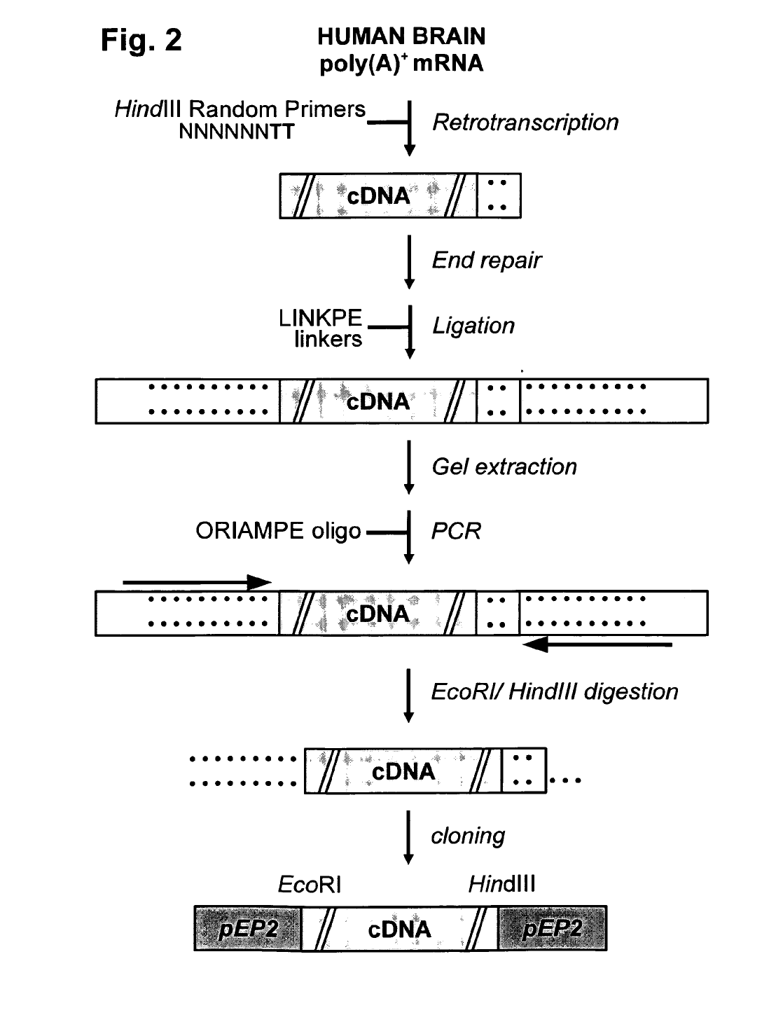

The scheme for constructing the phage display library from human brain is shown in FIG. 2. The first oriented strand of cDNA was synthesized starting from mRNA poly(A)+ of human brain obtained from BD Bioscience, in particular of a total of eight forebrains of healthy Caucasian males who died unexpectedly. The system for directed cloning of cDNA consists of using, during retrotranscription, oligonucleotides with a random sequence, but with a conserved sequence (TT) at 5′ (OrientExpress Random Primers Novagen). With an mRNA/oligonucleotide ratio of 1/2.5, cDNA fragments were obtained from 100 bp to 2 kb. Both the first and the second cDNA strand were synthesized in the presence of 5-methyl-dCTP to protect the internal restriction sites EcoRI and HindIII″ from subsequent digestions. Next, after ligation with specific linkers “EcoRI/HindIII” designed by us, for which a HindIII site is recreated only at the 3′ end owing to the two A's inserted in the synthesis of the first strand, the oriented cDNAs of length between 300 and 800 bp were purified with agarose gel, amplified, submitted to digestion with EcoRI and HindIII and cloned into pEP2. The ligation mixture was transformed into electrocompetent TOP10 F′ E. coli cells, then selected for the presence of ORF inserts by plating in the presence of 12 μg/ml of ampicillin. 1.35×10⁵ independent clones were obtained and the library was recombined to remove the β-lactamase gene (Zacchi et al., 2003). The average length, diversity and identity of the clones of the library were evaluated by PCR, restriction fingerprinting and random sequencing.

### Methodology

Construction of the Phage Display Library of Human Brain cDNA

The cDNA library was constructed from 1 μg of human brain poly(A)+ RNA (Clontech, cod. 6516-1). Synthesis of the first cDNA strand used 2.5 μg of Random Primers HindIII (Novagen), 200 U of SuperScriptlll RT (Invitrogen) and 200 U of RNaseOUT (Invitrogen), in the presence of methylated dNTPs 0.5 mM, according to the manufacturer's instructions. The second strand was synthesized with 23 U of DNA polymerase I (Promega) and 0.8 U of RNase H (USB). The reaction was incubated at 14° C. for 2 hours and purified with phenol:chloroform. After end repair by adding 1.5 U of T4 DNA polymerase (NEB), in the presence of 0.4 mM dNTP and incubation at 12° C. for 20 min, the cDNA was ligated to the linkers

### LINKPE

(LINKPE53 per 5′-AGGGGAGGGGGCTTGAATTCAAGC-3′ and LINKPE35 per 5′-CTCCCCT pGCTTGAATTCAAGCCCC-3′), pre-incubated at 95° C. for 2 min, 65° C. for 5 min and 42° C. for 10 min. The reaction was carried out at 16° C. overnight. The cDNA fragments in the range 300-800 bp were extracted and purified with agarose gel and amplified by the primers complementary to the linker sequence

ORIAMPEFOR (5′-GAGGGGGCTTGAATTCAAGC-3′) and

ORIAMPEREV (5′-GGGGGCTTGAATTCAAGCTT-3′).

PCR was carried out in the presence of 1 U of DNA polymerase Phusion Hot Start (Finnzymes) for 30 cycles in the following conditions: denaturation for 10 s at 98° C., pairing for 30 s at 60° C., extension for 45 s at 72° C. After purification, the cDNA fragments were digested, separately, with 30 U of HindIII (Promega) and with 30 U of EcoRI (NEB). Both reactions were incubated at 37° C. for 3 hours. The fragments were then mixed with the pEP2 vector, cut both with HindIII and EcoRI, in a molar ratio equal to 10:1 and ligated using T4 DNA ligase (NEB). The ligation to mixture was purified with phenol:chloroform and divided into 8 aliquots, each of which was used for transforming 40 μL of electrocompetent E. coli TOP10F′ cells (Invitrogen). The transformation mixture was plated on medium containing 25 μg/ml of cloramfenicol and 12 μg/ml of ampicillin. The resultant colonies were analysed by PCR, enzymatic fingerprinting and sequencing. After selection on ampicillin plates, the β-lactamase gene was removed by infecting BS1365 cells (which express Cre-recombinase constitutively) with the phages of the library in 10 ml of 2×TY, 25 μg/ml of kanamycin, 1% of glucose at 37° C. up to growth equal to OD of 0.5. After adding 25 μg/ml cloramfenicol, recombination took place during bacterial growth at 30° C. overnight. The next day, the bacteria were diluted 1/20 and were grown to OD of 0.5 at 37° C. After infection with the helper phage M13K07 at 37° C. for 30 min and growth at 30° C. overnight, the phages produced were used for infecting cells of E. coli DH5αF′. The clones obtained represent the library of selected ORF fragments.

### Example 5

### Selection of the Human Brain cDNA Library with the scFv Library from CSF of Patients with MS

Using the phage library of scFv from CSF of patients as coating of an immunotube, three selection cycles were performed, obtaining a 160-fold enrichment of specific clones.

From the last selection cycle, 94 clones selected at random were analysed by ELISA to evaluate their reactivity in comparison with the total soluble scFvs. 19 clones with an optical density (OD) above 0.2 were analysed by PCR; 17 of these had a correct insert and were sequenced. Sequence analysis revealed that all the clones were correctly “in-frame”. Some clones corresponded to superposed sequences of the same protein; the complete list of proteins identified as candidate autoantigens of MS are shown in Table 2; some of these constitute mimotopes. In particular, identification of the protein DDX24 will be revealed, as this same protein was identified in two other selections of the same human brain antigen library performed, respectively, with a set of sera and a set of CSF of RR patients.

TABLE 2
List of antigens identified in the procedure for selection of the antigen phage library with the antibody
library from CSF of patients with multiple sclerosis; the table shows the identifying acronyms
of the clones, the access number of the respective nucleotide sequence in the NCBI database,
description of the sequence coding for the antigen fragment of the clone, in the case of homology
at nucleotide level (cds) or of the protein homologous to the peptide encoded by the sequence
identified (mimotope), and the corresponding amino acids.

| | NCBI access | | | |
|---|---|---|---|---|
| Clones | number | Description | CDS | a.a. |
| HBscFvA1 | NM_020414.3 | Homo sapiens DEAD (Asp-Glu-Ala-Asp) box | real | 87-215 |
| HBscFvB10 | | polypeptide 24 (DDX24) | | |
| HBscFvE3 | | | | 99-215 |
| HBscFvA5 | | | | |
| HBscFvB5 | | | | 140-229 |
| HBscFvH5 | | | | |
| HBscFvB7 | NM_024863.4 | Homo sapiens transcription elongation factor A | real | 98-148 |
| | | (SII)-like 4 (TCEAL4), transcript variant 1 | | |
| HBscFvE5 | NM_006706.3 | Homo sapiens transcription elongation regulator 1 | real | 683-793 |
| | | (TCERG1), transcript variant 1 | | |
| HBscFvE8 | NM_174889.2 | Homo sapiens NDUFA12-like (NDUFA12L) | real | 1-118 |
| HBscFvG2 | NM_002128.3 | Homo sapiens high-mobility group box 1 (HMGB1) | real | 7-95 |
| HBscFvG6 | gbAAC51279.1 | Homo sapiens Putative p150 (RT like) | mimotope | 481-500 |
| HBscFvG7 | gbABX35540.1 | DEAD/DEAH box helicase domain protein | mimotope | 452-531 |
| | | (Delftia acidovorans) | | |
| HBscFvC12 | dbjBAD93163.1 | caveolin 1 variant [Homo sapiens] | mimotope | 30-45 |
| HBscFvA8 | | | | |
| HBscFvH11 | GENE ID: | Flotillin-2 [Homo sapiens] | mimotope | 28-38 |
| HBscFvB6 | 2319FLOT2 | | | |
| HBscFvF4 | refXP_729762.1 | Senescence-associated protein | mimotope | 144-158 |
| | | [Plasmodium yoelii] | | |

### Methodology

Selections between cDNA and scFv libraries

For each cycle, a well of a microtitre plate (Costar) was coated with 9×10¹² phages of the scFv library in carbonate buffer (NaHCO₃ 1M pH 9) at 4° C. overnight. After washing with PBS, saturation was carried out for 1 hour at room temperature with BSA 3% in PBS.

The phages of the cDNA library, purified by means of PEG, were diluted in an equal volume of BSA 6% in PBS, incubated for 30 minutes at room temperature, added to the well and incubated for 30 min with stirring and a further 90 min, still at room temperature.

For the first cycle, 10 washings were performed with PBS-Tween (PBST) 0.5% and 10 with PBS. The phages were eluted with glycine 200 mM pH 2.2 in BSA 2%, neutralized with Tris-HCl 1M pH 9 and used for infecting E. coli DH5αF′ cells, grown to OD of 0.5, for 40 min at 37° C. The phages were recovered by infection with the helper phage M13K07, in 10 ml of 2xYT with 25 μg/ml cloramfenicol and 25 μg/ml kanamycin, and incubation at 30° C. overnight.

The procedure was similar for the other cycles, except that for the second cycle, 10 washings were performed with PBST 0.1% and 10 with PBS, while for the third cycle there were 20 washings with PBST 0.1% and 20 with PBS. For monitoring the enrichment of specific clones, the amount of phages selected at input and output for each cycle was titrated and the input/output ratio was determined. After selection, 95 individual clones selected at random were submitted to phage-ELISA analysis.

### Example 6

### The Protein TCERG1 as Putative Autoantiqen of MS

Starting from the protein TCERG1 (SEQ ID NO. 1), the sequence according to the present invention comprises a portion of protein TCERG1 (otherwise known as CA150 or TAF2S) corresponding to the amino acids from position 677 to 1098 (SEQ ID NO. 2) and respective possible modifications as deducible from the annotations present in public databases, available from the sites given below and summarized hereunder, and from the definition of a consensus sequence given above.

Access codes of the protein:

### HGNC:15630

### Ensembl:ENSG00000113649

### HPRD:10393

### REFERENCE SITES

http://www.ncbi.nlm.nih.qov/nuccore?Db=qene&Cmd=retrieve&dopt=full report&list_uids=10915&loq$=databasead&loqdbfrom=nuccore
http://www.qenenames.orq/data/hqnc data.php?hqnc id=15630
http://www.hprd.orq/summary?hprd_id=10393 &isoform id=10393 1 &isoform name=Isoform 1

| | |
|---|---|
| 677 | AFST WEKELHKIVF DPRYLLLNPK ERKQVFDQYV KTRAEEERRE KKNKIMQAKE DFKKMMEEAK |
| 741 | FNPRATFSEF AAKHAKDSRF KAIEKMKDRE ALFNEFVAAA RKKEKEDSKT RGEKIKSDFF ELLSNHHLDS QSRWSKVKDK |
| 821 | VESDPRYKAV DSSSMREDLF KQVIEKIAKN LDSEKEKELE RQARIEASLR EREREVQRAR SEQTKEIDRE REQHKREEAI |
| 901 | QNFKALLSDM VRSSDVSWSD TRRTLRKDHR WESGSLLERE EKEKLFNEHI EALTKKKREH FRQLLDETSA ITLTSTWKEV |
| 981 | KKIIKEDPRC IKFSSSDRKK QREFEEYIRD KYITAKADFR TLLKETKFIT YRSKKLIQES DQHLKDVEKI LQNDKRYLVL |
| 1061 | DCVPEERRKL IVAYVDDLDR RGPPPPPTAS EPTRRSTK |

### Alternative Names

transcription elongation regulator 1
TATA box binding protein (TBP)-associated factor, RNA polymerase II, S, 150 kD
TATA box binding protein associated factor 2S
Transcription factor CA150
co-activator of 150 kDa Complete sequence corresponding to the two isoforms 1 and 2 (access code: NP_{—}006697 and NP_{—}001035095)

SEQ ID NO: 2
AFSTWEKELHKIVFDPRYLLLNPKERKQVFDQYVKTRAEEERREKKNKIM
QAKEDFKKMMEEAKFNPRATFSEFAAKHAKDSRFKAIEKMKDREALFNEF
VAAARKKEKEDSKTRGEKIKSDFFELLSNHHLDSQSRWSKVKDKVESDPR
YKAVDSSSMREDLFKQYIEKIAKNLDSEKEKELERQARIEASLREREREV
QKARSEQTKEIDREREQHKREEAIQNFKALLSDMVRSSDVSWSDTRRTLR
KDHRWESGSLLEREEKEKLFNEHIEALTKKKREHFRQLLDETSAITLTST
WKEVKKIIKEDPRCIKFSSSDRKKQREFEEYIRDKYITAKADFRTLLKET
KFITYRSKKLIQESDQHLKDVEKILQNDKRYLVLDCVPEERRKLIVAYVD
DLDRRGPPPPPTASEPTRRSTK

Single nucleotide polymorphisms (SNPs) present in the region
rs12186370: SNP synonym (G/A), corresponding to the third base of the codon coding for the a.a. Thr [T] in position 976;
rs4705103: SNP synonym (G/A), corresponding to the third base of the codon coding for the a.a. Ser [S] in position 1040; Consensus sequence between two antigens

The following consensus sequence was constructed taking into account the homology of the antigen TCERG1 isolated SEQ ID NO:3, included in the sequence of the human TCERG1 protein and corresponding to the a.a. 683-793, with another antigen, DDX24, identified in the selection described and characterized by a high reactivity to biological samples from patients with MS. The alternative amino acids are indicated in the column.

| | |
|---|---|
| 683 | KELHKIVF DPRYLLLNPK ERKQVFDQYV KTRAEEERRE KHNKIMQAKE DFKKMMEEAK |
| | KK [symbol] G GL |
| 741 | FNPRATFSEF AAKHAKDSRF KAIEKMKDRE ALFNEFVAAA RKKEKEDSKT RGE |
| | VP |

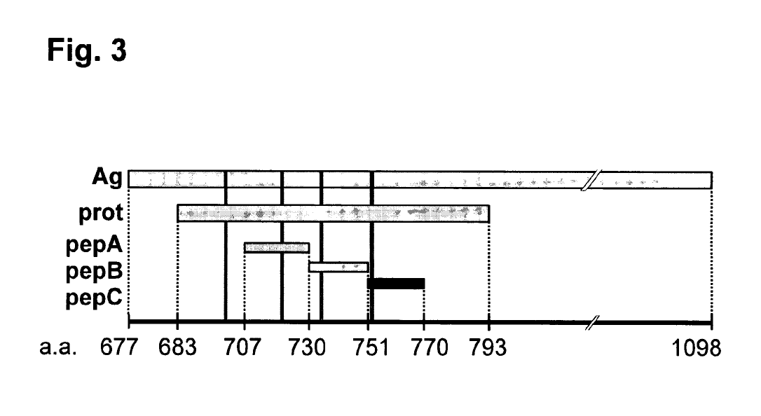

FIG. 3 shows the portions of maximum homology between the two proteins TCERG1 and DDX24 (from 57 to 67%); bearing in mind that these two antigens were found to be more frequent in the selections of the phage library with the patients' antibodies, these portions might correspond to epitopes that are more recognized, around which peptides could be synthesized, to be used in diagnostic tests.

The consensus sequence of the invention of polypeptide SEQ ID NO. 2 takes account of the regions of homology with another antigen identified, as illustrated above, and is shown below.

| | |
|---|---|
| 577 | AFST WEKELHKIVF DPRYLLLNXX XXXQVFDQYV KTRAEEERXX XXXXXMQAKE DFXXXXXEAK |
| | PK ERK RE KKNKI KKMME |
| | KK [symbol] G GL |
| 741 | FNPRATESEF XXXXXXDSRF KAIEKMKDRE ALFNEFVAAA RKKEKEDSKT RGEKIKSDFF ELLSNHHLDS QSRWSKVKDK |
| | AAKHAK |
| | VP |
| 821 | VESDPRYKAV DSSSMREDLF KQYIEKIAKN LDSEKEKELE RQARIEASLR EREREVQKAR SEQTKEIDRE REQHKREEAI |
| 901 | QNFKALLSDM VRSSDVSWSD TRRTLRKDHR WESGSLLERE EKEKLFNEHI EALTKKKREH FRQLLDETSA ITLTSTWKEV |
| 981 | KKIIKEDPRC IKFSSSDRKK QREFEKYIRD KYITAKADFR TLLKETKFIT YRSKKLIQES DQHLKDVEKI LQNDKRYLVL |
| 1061 | DCVPEERRKL IVAYVDDLDR RGPPPPPTAS EPTRRSTK |

### Example 7

### Production of the Recombinant Protein

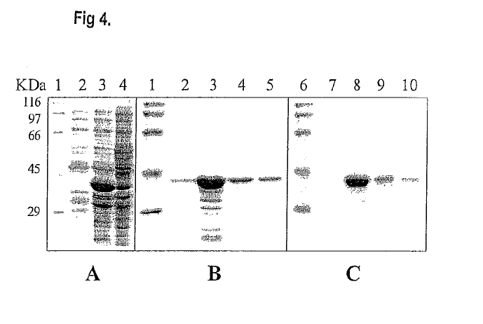

The cDNA coding for a portion of the human protein TCERG1, corresponding to the amino acids 677-1098 and comprising the epitope identified (a.a. 683-793), was cloned in the pASK45 expression vector (IBA). The cDNA was obtained by PCR from human brain cDNA using oligonucleotides designed on the sequence of the corresponding mRNA. Expression took place in cells of E. coli Rosetta 2, after optimization of the induction times. The protein was purified by affinity chromatography in two successive passes to obtain the complete form (with two purification tags at the ends) and at higher degree of purity (FIG. 4).

### Production of Recombinant Antigens

The cDNA of TCERG was obtained by PCR from human brain total cDNA using specific primer designed on the basis of the sequences available in the database:

TCERG For
(5′-AAAATTCAGCTTTGATTTCAACGTGGGAGAAG-3′)
and
TCERG Back
(5′-TTCCTGCAGCCTTTTGTTGATGTGCTCCGTGG-3′).

The cDNA was purified from gel, digested successively with EcoRI and PstI to (NEB), ligated to the vector pASK-45plus (IBA) and transformed into Rosetta2 cells.

The recombinant protein rTCERG (677-1098) was induced by a clone containing the vector TCERG (677-1098)-pASK45plus with anhydrotetracycline 200 ng/ml for 3 hours at 37° C. The bacterial pellet was resuspended and the protein was purified as described in the manufacturer's protocol for purification by Strep-tagII (IBA). The protein was further purified using the second tag at the C-terminal. The sample was diluted 1:10 in solution A (20 mM Tris-HCl pH 8, 50 mM NaCl, 5 mM imidazole) and loaded on a column NiNTA (Amersham) equilibrated with the same buffer. After washing with 15 ml of solution B (20 mM Tris-HCl pH 8, 50 mM NaCl, 0.1% Triton X-100 (v/v), 20 mM imidazole) and 10 ml of solution A, the sample was eluted with 10 ml of elution buffer (20 mM Tris-HCl pH 8, 50 mM NaCl, 300 mM imidazole). All the aliquots eluted were checked by electrophoresis.

### Example 8

### Synthesis of Peptides

Three peptides of 21-23 amino acids, corresponding to three different, superposed portions (FIG. 3) of the TCERG1 antigen identified (peptide TCERG1-A: a.a. 707-729 (SEQ ID NO. 5); peptide TCERG1-B: a.a. 730-750 (SEQ ID NO. 6); peptide TCERG1-C: a.a. 751-770) (SEQ ID NO. 4), were synthesized (Gen Way Biotech, Inc), adopting the following criteria: net charge different from 0, percentage of acidic or basic amino acids greater than 25% and of hydrophobic amino acids less than 25%, absence of cysteines (to avoid formation of any disulphide bridges).

### Example 9

### Primary Phage-ELISA

Selected single clones were cultured in a titration plate with 96 round-bottomed wells up to OD of 0.5. Each clone was infected with a helper phage M13K07 at 37° C. for 30 min and was incubated at 30° C. overnight to allow production of the phages. In parallel, 96 flat-bottomed wells of a titration plate (Costar) were covered by incubation overnight at 4° C. with the soluble form of the scFvs of the antibody library from patients' cerebrospinal fluid and were saturated with BSA 2% in PBS for 1 hour at room temperature. Then each supernatant of the phage cultures was diluted 1:1 (v/v) in BSA 4%-PBS and incubated for 90 min at room temperature. After 3 washings with PBST 0.1% and 3 with PBS, an anti-M13 monoclonal antibody, conjugated with peroxidase (Amersham Pharmacia Biotech), diluted 1:3000 in BSA 2%-PBS, was added and incubated for 1 hour at room temperature. After 3 washings with PBST 0.1% and 3 with PBS, a colorimetric reaction was started with 3,3′,5,5′-tetramethylbenzidine (TMB, Sigma) and the plates were read at 450 nm.

Phage helper M13K07 was used as internal negative control. The immunoreactivity was measured for each phage clone as the OD ratio between the sample and the helper phage.

### Analysis of the Validity of TCERG1 as Biomarker of MS

For confirming the recognition and specificity of the TCERG1 antigen, secondary ELISA assays were performed using single samples of serum or CSF of patients with MS and other neurological diseases (OND) as well as from healthy donors.

The following assays (Table 3) were performed with the antigen according to the present invention in three different formats:

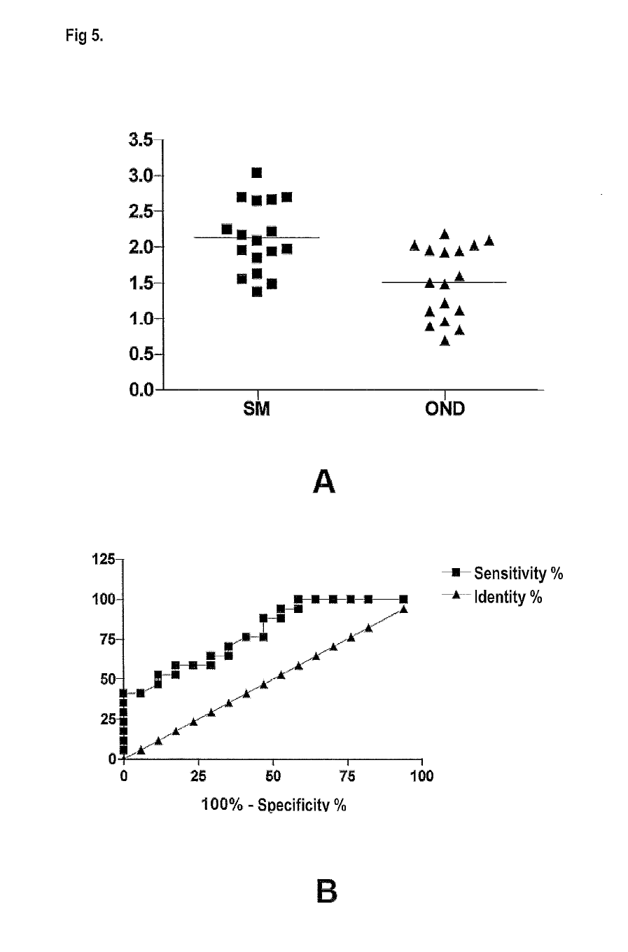

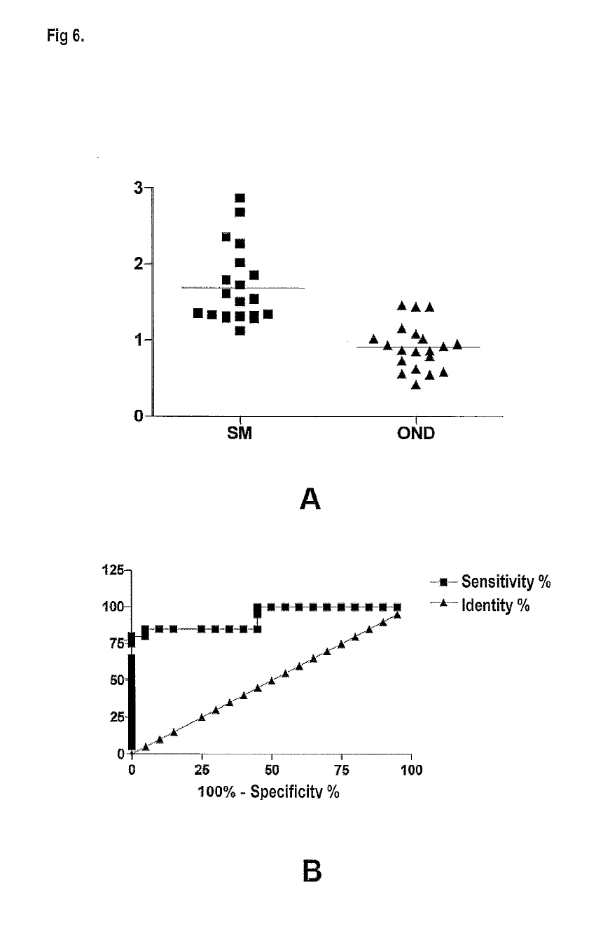

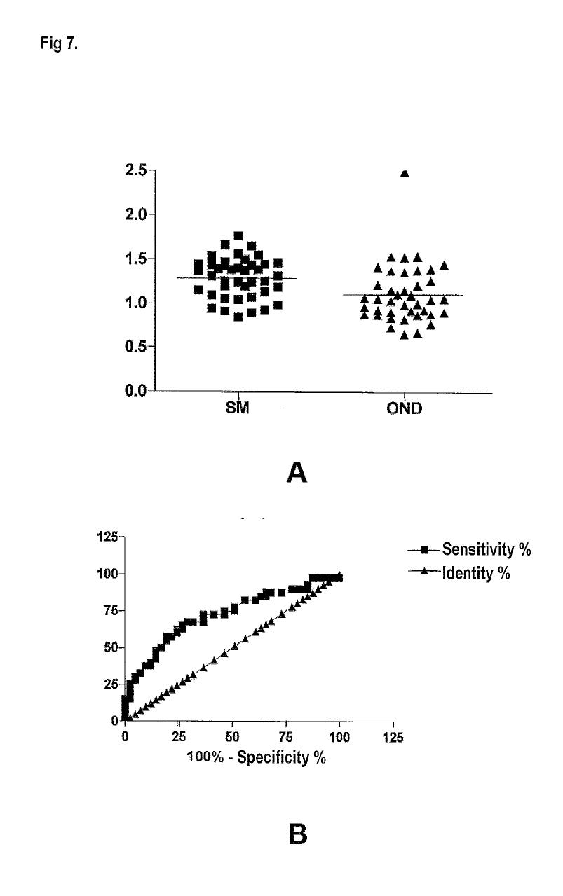

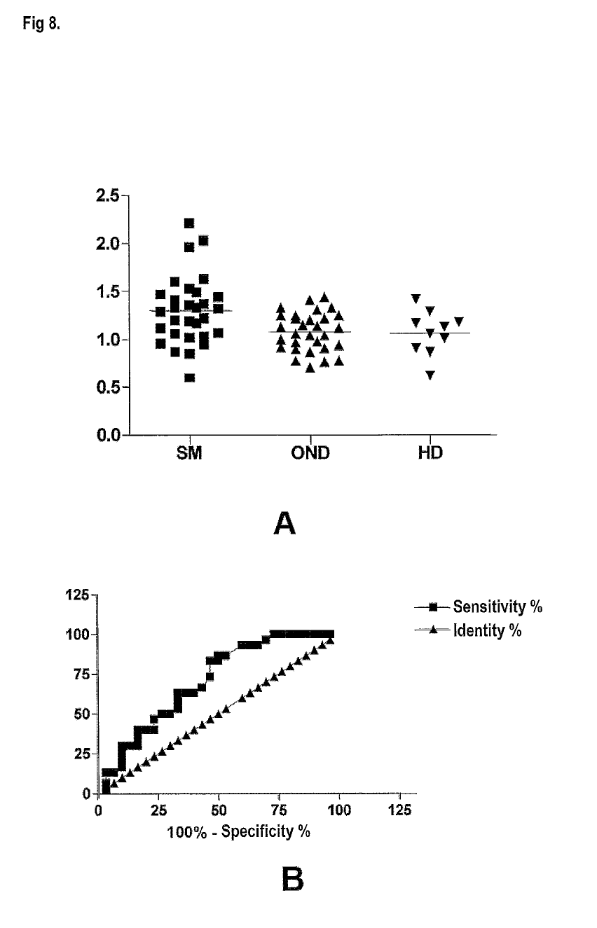

a) protein fragment corresponding to amino acids 683-793 of human TCERG1 (peptide TCERG1-A: SEQ ID NO. 5) exposed on the surface of a phage M13 (assay 1);
b) recombinant protein corresponding to amino acids 677-1098 of human TCERG1 (peptide TCERG1-B: SEQ ID NO. 6) (assay 2);
c) peptide corresponding to amino acids 751-770 (peptide TCERG1-C SEQ ID NO. 4) of human TCERG1 (assays 3 and 4): this peptide showed greater reactivity to serum samples from patients with MS in preliminary tests in which all three peptides TCERG1-A, B and C were assayed individually.
(1) “phage ELISA” on 17 CSF samples from patients with MS and 17 with OND: the two groups were found to be statistically different (p=0.0009); ROC analysis for assessing the diagnostic value of TCERG1 gave a likelihood ratio (LR) above 8, corresponding to a specificity of 94% and a sensitivity of 47% for a cut-off value equal to 2.12. Subjects who were true positives (PPV) and true negatives (NPV) were, respectively, 89% and 64% (FIG. 5).
(2) ELISA on 20 serum samples from patients with MS and 20 with OND D: the two groups were found to be statistically different (p<0.0001); ROC analysis for assessing the diagnostic value of TCERG1 gave a likelihood ratio (LR) above 11, corresponding to a specificity of 95% and a sensitivity of 55% for a cut-off value equal to 1.45. Subjects who were true positives (PPV) and true negatives (NPV) were, respectively, 92% and 68% (FIG. 6).
(3) ELISA on 41 serum samples from patients with MS and 40 with OND: the two groups were found to be statistically different (p=0.01); ROC analysis for assessing the diagnostic value of TCERG1 gave a likelihood ratio (LR) above 2.22, corresponding to a specificity of 90% and a sensitivity of 22% for a cut-off value equal to 1.45. Subjects who were true positives (PPV) and true negatives (NPV) were, respectively, 69% and 53% (FIG. 7).
(4) ELISA on 30 serum samples from patients with MS, 40 with OND and 10 healthy subjects: the three groups were found to be statistically different (p=0.01); ROC analysis for assessing the diagnostic value of TCERG1 gave a likelihood ratio (LR) above 12, corresponding to a specificity of 98% and a sensitivity of 30% for a cut-off value equal to 1.44. Subjects who were true positives (PPV) and true negatives (NPV) were, respectively, 90% and 65% (FIG. 8).

TABLE 3
Synopsis of the statistical data for the various secondary ELISA assays carried out using single samples of serum
or CSF of patients with multiple sclerosis (MS), other neurological diseases (OND) and from healthy donors (HD).
The cut-off represents the threshold value of the optical density ratio, obtained for each sample in the presence
and absence of antigen, used for calculating the values of specificity, sensitivity, PPV, NPV and LR+.

| | | | | | | | | | | | | Significance | |
|---|---|---|---|---|---|---|---|---|---|---|---|---|---|
| Assay | Antigen | Samples | MS | Ctrl | HD | cut-off | Specificity | Sensitivity | PPV | NPV | LR+ | (P) | Area ROC |
| 1 | phage | CSF | 17 | 17 | 0 | 2.12 | 94% | 47% | 89% | 64% | 8 | 0.0009 | 0.8 |
| 2 | prot | sera | 20 | 20 | 0 | 1.45 | 95% | 55% | 92% | 68% | 11 | <0.0001 | 0.93 |
| 3 | peptide | sera | 41 | 40 | 0 | 1.45 | 90% | 22% | 69% | 53% | 2.2 | 0.01 | 0.72 |
| 4 | peptide | sera | 30 | 40 | 10 | 1.44 | 98% | 30% | 90% | 65% | 12 | 0.01 | 0.71 |

### Secondary Phage-ELISA

The wells of a titration plate with 96 flat-bottomed wells (Costar) were covered by incubation overnight at 4° C. with the antibodies present in the serum (diluted 1:100 in PBS) and in the CSF (diluted 1:5 in PBS) of individual patients or healthy donors. After saturation with BSA 2% in PBS for 1 hour at room temperature, 10⁹ or 10¹⁰ phages, precipitated with PEG, of a single clone selected in the human brain library, diluted in BSA 2% in PBS, were added and incubated for 1 hour at room temperature. The protocol was continued as in primary Phage-ELISA.

For each sample, a helper phage M13K07 was used as negative control and the immunoreactivity was measured as the OD ratio between the phage tested and the helper phage.

### Example 10

### Test of Specificity of the TCERG1 Antigen

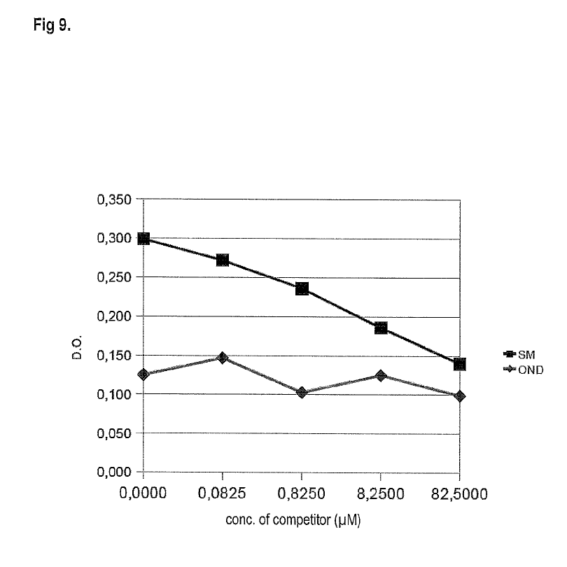

In order to confirm the specific recognition of the peptide TCERG1-C by the sera from MS, a competitive ELISA assay was performed. One of the MS sera that proved statistically “true positive” and one of the OND sera statistically “true negative” were pre-incubated with increasing concentrations of peptide TCERG1-C (from 0 to 82.5 μM). After incubation for 30 minutes, the reactivity of the serum was evaluated by ELISA using the same peptide. Effective, progressive competition was observed in the case of pre-incubation of the MS serum with the peptide, whereas this is absent in the case of the OND serum (FIG. 9). In fact, in the case of the MS serum, at the higher competitor concentration, the value of OD is similar to that of the control sample, which remains almost constant, demonstrating a specificity of recognition of the peptide TCERG1-C by the MS serum.

ELISA with the Recombinant Antigen

Using the recombinant protein as antigen, ELISA was performed by coating each well of the microtitre plate with 1 μg of the protein produced by incubation overnight at 4° C. An equivalent number of wells were incubated with only the buffer used for resuspending the proteins, as negative control of the reaction. After saturation with BSA 2% in PBS for 1 hour at room temperature, a different biological sample was added to each well (serum diluted 1:100 or CSF diluted 1:5 in BSA 2% in PBS) and was left for 1 hour at room temperature. After 3 washings with PBST 0.1% and 3 with PBS, an anti-human-IgG antibody, conjugated with peroxidase (Dako), diluted 1:1000 in BSA 2%-PBS, was added and incubated for 1 hour at room temperature. After 3 washings with PBST 0.1% and 3 with PBS, a colorimetric reaction was started with 3,3′,5,5′-tetramethylbenzidine (TMB, Sigma) and the plates were read at 450 nm.

The immunoreactivity of each sample was evaluated as the ratio of OD detected with the antigen present or absent.

ELISA with Peptides

The ELISA performed using single peptides as antigen is identical to that in which whole recombinant protein is used, except that special plates were used, functionalized for attachment of the peptides: Reacti-Bind™ plates (Pierce). Each well was coated with 2 μg of peptide, whereas no peptide was added in the negative controls. The protocol is similar to that described above.

In competitive ELISA, the biological samples to be tested were a serum of a patient with MS and that of a patient with another neurological disease; both were pre-incubated with increasing concentrations of the same peptide used for coating the wells (0 M; 0.0825 M; 0.825 M; 8.25 M; 82.5 M), for 30 min at room temperature. Next, the reactivity of the peptide was determined as described above.

### Statistical Analysis

Statistical analysis was performed using the software GraphPad Prism version 4.0. The significance of the differences between the groups of patients was evaluated using the t-test for unpaired data (one-sided), in the case of normal distributions, or the Mann-Whitney test (one-sided), if normal distribution is absent for at least one of the two groups; in the case of three groups, an ANOVA analysis was carried out. A value of p<0.05 was regarded as statistically significant. ROC analysis was carried out for determining the threshold values, of sensitivity and specificity, and of LR+.

What is claimed is:

1. A method for the diagnosis of multiple sclerosis, comprising the step of detecting a protein selected from the group consisting of: SEQ ID NO. 2, SEQ ID NO. 3 and SEQ ID NO. 4.

2. The method according to claim 1, wherein said protein is a biological marker for monitoring the efficacy of the therapeutic treatment of multiple sclerosis.

3. The method according to claim 1, wherein said protein is a biological marker for monitoring inflammatory state parameters, after by interferon treatment.

4. The method according to claim 1, wherein said protein is a biological marker for distinguishing patients with multiple sclerosis from patients with other neurological diseases such as polyneuropathies, polyneuritis, polyradiculoneuritis, encephalitis, myelitis, meningitis, leukoencephalopathies, vasculitis, Miller Fisher's Guillain Barrè's syndromes, amyotrophic lateral sclerosis, spastic tetraparesis, paraneoplastic neuropathies, Charcot Marie Tooth's syndrome, spinal cord injuries, hydrocephalus, subaracnoidea hemorrhages.

5. The method according to claim 1, wherein said protein is a biological marker in protein platforms (array).

6. The method according to claim 5, wherein said protein platforms are protein chips.

7. The method according to claim 1, wherein said protein is a biological marker for determining the individual immunological profiles.

8. The method according to claim 1, wherein said protein is a biological marker for discriminating between different states of multiple sclerosis.

9. The method according to claim 8, wherein said states are selected for the group consisting of:
a. relapsing-remitting form (RR);
b. primary form; and
c. secondary-progressive form.

10. (canceled)

11. The method according to claim 1, comprising the steps of:
a. providing a biological sample;
b. providing any one of the proteins selected from the group consisting of SEQ ID NO:2, SEQ ID NO:3 and SEQ ID NO:4 immobilized onto a solid substrate;
c. contacting the sample from step a. with the substrate from step b.;
d. detecting the reaction.

12. The method according to claim 11, wherein said step d. is carried out by means of a technique selected from the group consisting of:
a. ELISA-type assays;
b. radioimmunological-type (RIA) assays;
c. immunological-type (Western Blot and LINE blot) assays;
d. protein microarrays assays.

13. The method according to any one of claim 11, for discriminating between patients with neurological symptoms and patients with multiple sclerosis.

14. The method according to claim 11, wherein said biological sample is a serum or liquor sample.

15. Diagnostic kit comprising any one of the proteins selected from the group consisting of SEQ ID NO:2, SEQ ID NO:3 and SEQ ID NO:4, which serves as an antigen and a reagent for separate, simultaneous and consecutive use, for diagnosing an autoimmune disease.

16. Diagnostic kit according to claim 15, wherein said autoimmune disease is multiple sclerosis.

17. A method for detecting autoantigens in multiple sclerosis, comprising the steps of:
a. preparing a phage display library of scFv antibodies from the cerebrospinal fluid of one or more patients with multiple sclerosis;
b. preparing a phage display library enriched in ORF cDNA fragments from human brain;
c. selecting the library of step b with the library of step a, said selection being carried out by interacting the immobilized antibodies phage library onto an immunotube with the antigen phage library to the same phage titer;
d. sequencing the clones selected in step c.


SEQUENCE LISTING (6 sequences)

SEQ ID NO: 1 - protein, 1098 aa, Homo sapiens
  SOURCE 1..1098: /mol_type=“protein” /organism=“Homo sapiens”
MAERGGDGGESERFNPGELRMAQQQALRFRGPAPPPNAVMRGPPPLMRPPPPFGMMRGPPPPPRPPFGRPPFDPNMPPMPPPGGIPPPMGPPHLQRPPFMPPPMSSMPPPPGMMFPPGMPPVTAPGTPALPPTEEIWVENKTPDGKVYYYNARTRESAWTKPDGVKVIQQSELTPMLAAQAQVQAQAQAQAQAQAQAQAQAQAQAQAQAQAQAQAQAQAQAQAQAQAQAQAQAQAQAQAQAQVQAQVQAQVQAQAVGASTPTTSSPAPAVSTSTSSSTPSSTTSTTTTATSVAQTVSTPTTQDQTPSSAVSVATPTVSVSTPAPTATPVQTVPQPHPQTLPPAVPHSVPQPTTAIPAFPPVMVPPFRVPLPGMPIPLPGVAMMQIVSCPYVKTVATTKTGVLPGMAPPIVPMIHPQVAIAASPATLAGATAVSEWTEYKTADGKTYYYNNRTLESTWEKPQELKEKEKLEEKIKEPIKEPSEEPLPMETEEEDPKEEPIKEIKEEPKEEEMTEEEKAAQKAKPVATAPIPGTPWCVVWTGDERVFFYNPTTRLSMWDRPDDLIGRADVDKIIQEPPHKKGMEELKKLRHPTPTMLSIQKWQFSMSAIKEEQELMEEINEDEPVKAKKRKRDDNKDIDSEKEAAMEAEIKAARERAIVPLEARMKQFKDMLLERGVSAFSTWEKELHKIVFDPRYLLLNPKERKQVFDQYVKTRAEEERREKKNKIMQAKEDFKKMMEEAKFNPRATFSEFAAKHAKDSRFKAIEKMKDREALFNEFVAAARKKEKEDSKTRGEKIKSDFFELLSNHHLDSQSRWSKVKDKVESDPRYKAVDSSSMREDLFKQYIEKIAKNLDSEKEKELERQARIEASLREREREVQKARSEQTKEIDREREQHKREEAIQNFKALLSDMVRSSDVSWSDTRRTLRKDHRWESGSLLEREEKEKLFNEHIEALTKKKREHFRQLLDETSAITLTSTWKEVKKIIKEDPRCIKFSSSDRKKQREFEEYIRDKYITAKADFRTLLKETKFITYRSKKLIQESDQHLKDVEKILQNDKRYLVLDCVPEERRKLIVAYVDDLDRRGPPPPPTASEPTRRSTK

SEQ ID NO: 2 - protein, 422 aa, Homo sapiens
  SOURCE 1..422: /mol_type=“protein” /organism=“Homo sapiens”
AFSTWEKELHKIVFDPRYLLLNPKERKQVFDQYVKTRAEEERREKKNKIMQAKEDFKKMMEEAKFNPRATFSEFAAKHAKDSRFKAIEKMKDREALFNEFVAAARKKEKEDSKTRGEKIKSDFFELLSNHHLDSQSRWSKVKDKVESDPRYKAVDSSSMREDLFKQYIEKIAKNLDSEKEKELERQARIEASLREREREVQKARSEQTKEIDREREQHKREEAIQNFKALLSDMVRSSDVSWSDTRRTLRKDHRWESGSLLEREEKEKLFNEHIEALTKKKREHFRQLLDETSAITLTSTWKEVKKIIKEDPRCIKFSSSDRKKQREFEEYIRDKYITAKADFRTLLKETKFITYRSKKLIQESDQHLKDVEKILQNDKRYLVLDCVPEERRKLIVAYVDDLDRRGPPPPPTASEPTRRSTK

SEQ ID NO: 3 - protein, 111 aa, Homo sapiens
  SOURCE 1..111: /mol_type=“protein” /organism=“Homo sapiens”
KELHKIVFDPRYLLLNPKERKQVFDQYVKTRAEEERREKKNKIMQAKEDFKKMMEEAKFNPRATFSEFAAKHAKDSRFKAIEKMKDREALFNEFVAAARKKEKEDSKTRGE

SEQ ID NO: 4 - protein, 20 aa, Homo sapiens
  SOURCE 1..20: /mol_type=“protein” /organism=“Homo sapiens”
AAKHAKDSRFKAIEKMKDRE

SEQ ID NO: 5 - protein, 23 aa, Homo sapiens
  SOURCE 1..23: /mol_type=“protein” /organism=“Homo sapiens”
DQYVKTRAEEERREKKNKIMQAK

SEQ ID NO: 6 - protein, 21 aa, Homo sapiens
  SOURCE 1..21: /mol_type=“protein” /organism=“Homo sapiens”
EDFKKMMEEAKFNPRATFSEF